(19) United States
(12) Patent Application Publication
(10) Pub. No.: US 2019/0210016 A1
(43) Pub. Date: Jul. 11, 2019

(54) DUTY CYCLE OPTIMIZATION IN SINGLE-MOLECULE DETECTION

(71) Applicant: Genturi, Inc., WOBURN, MA (US)

(72) Inventors: Qun ZHONG, Lexington, MA (US); Jeff KROGMEIER, Winchester, MA (US); Andrew GOLDEN, Boston, MA (US); Samrudhi SHARMA, Billerica, MA (US); Laurent MENARD, Raleigh, NC (US); Andrew WATSON, Bedford, MA (US); Oscar MCCRATE, Detroit, MI (US); Jun ZHOU, Belmont, MA (US); Rachel ROWELL, Salem, MA (US)

(21) Appl. No.: 16/238,986

(22) Filed: Jan. 3, 2019

Related U.S. Application Data
(60) Provisional application No. 62/613,966, filed on Jan. 5, 2018.

(51) Int. Cl. B01L 3/00; C12Q 1/68; G01N 27/447

(52) U.S. Cl. CPC B01L 3/5027; B01L 3/50273; C12Q 1/68; G01N 27/44791; G01N 27/44704; B01L 2400/0403; B01L 2400/0409; B01L 2400/0487; B01L 2400/0418; B01L 2300/0645; B01L 2400/0421; B01L 2300/0896; B01L 2300/0861; B01L 2400/049

(57) ABSTRACT

A fluidic chip for detecting an analyte is provided, which comprises (a) at least one device comprising (i) a fluid inlet and (ii) at least one fluid transport channel that passes through a detection region, wherein (1) the at least one fluid transport channel has varying cross-sectional area as a function of position along the channel length and/or (2) the device comprises two or more fluid transport channels configured with two or more different cross-sectional areas; and (b) at least one fluid outlet, wherein the device provides for transport of single molecules across the detection region at a desired duty cycle by (1) allowing for detection of an analyte at a different position along the channel length based on the desired cross-sectional area and/or (2) allowing for detection of an analyte in a specific channel configured with the desired cross-sectional area. This application also provides for method of optimizing duty cycle using fluidic chips.

### CROSS-REFERENCE TO RELATED APPLICATIONS

This application claims the benefit of priority of U.S. Provisional Application No. 62/613,966, filed Jan. 5, 2018, the content of which is incorporated by reference herein in its entirety for any purpose.

### FIELD

Fluidic chips for detecting analytes and methods of use

### BACKGROUND

Fluidic chips, including nanofluidic chips, are used for a variety of lab-on-a-chip assays in the biotechnology field. In the research tools industry, customers desire short experimental run times on nanofluidic chips, as well as high quality results. In some nanofluidic chip systems, such as single-molecule detection systems, these two characteristics may conflict—for example, high quality results may require sufficient data points from which to estimate population parameters of the sample of DNA, while short experimental runs may lead to fewer data points because detection of single molecules may require adequate temporal spacing from molecule to molecule.

Furthermore, in some use cases, there may be a degree of uncertainty in the concentration of particular DNA species (analytes of interest and/or interferents) in the sample that is to be analyzed. Moreover, there may be uncertainty in the degree of data population overlap (e.g., size overlap of two or more populations of DNA) in a single sample, adding uncertainty to number of data points needed for sizing mean and resolving DNA populations. Either or both could then lead to uncertainty in necessary run time to attain high quality data.

The customer may also or alternatively choose to indicate a preference regarding the balance between quality and run time (i.e., duty cycle), which may vary from run to run.

Therefore, there is a need for accommodating a range of initial DNA concentrations and/or DNA population types or mixtures, and/or accommodating experimental run feedback in optimizing the duty cycle for quality and run time.

### SUMMARY

In accordance with the description, a fluidic chip for detecting an analyte comprises (a) at least one device comprising (i) a fluid inlet and (ii) at least one fluid transport channel that passes through a detection region, wherein (1) the at least one fluid transport channel has varying cross-sectional area as a function of position along the channel length and/or (2) the device comprises two or more fluid transport channels configured with two or more different cross-sectional areas; and (b) at least one fluid outlet, wherein the device provides for transport of single molecules across the detection region at a desired duty cycle by (1) allowing for detection of an analyte at a different position along the channel length based on the desired cross-sectional area and/or (2) allowing for detection of an analyte in a specific channel configured with the desired cross-sectional area.

In some embodiments a method of detecting an analyte using a fluidic chip comprises: (a) providing any of the fluidic chips described herein; wherein (1) if the at least one fluid transport channel has varying cross-sectional area as a function of position along the channel length, detecting an analyte at a different position along the channel length based on the desired cross-sectional area in order to optimize duty cycle, and/or (2) if the device comprises two or more channels configured with two or more different cross-sectional areas, detecting an analyte in a specific channel configured with the desired cross-sectional area in order to optimize duty cycle.

In some embodiments, a method of detecting an analyte using a fluidic chip comprises: (a) providing a fluidic chip comprising (i) at least one device comprising a fluid inlet and at least one fluid transport channel that passes through a detection region and (ii) a fluid outlet; (b) adjusting the rate of transport of single molecules through the fluid transport channel across at least the detection region by: (i) adjusting the actuation energy applied to the analyte; electro-osmotic force tuning; and (iii) tuning of ionic strength and/or mobility, wherein adjusting the rate of transport of single molecules yields a desired duty cycle.

Additional advantages will be set forth in part in the description which follows, and in part will be obvious from the description, or may be learned by practice. The advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 provides a graph showing experimental data of a DNA mixture comprising 5 kbp sized DNA. Electrophoretic-based throughput increases as nanochannel width increases, at a rate of x², where x² is proportional to the cross-sectional area of the channel. In the graph, width approximately equals depth. There is approximately a 25-fold increase in % molecules from 200 nm to 1000 nm width/depth channels.

FIG. 2 shows mean values of coefficients of variation (CVs) (y-axis) according to the channel size (x-axis), illustrating that DNA sizing precision (CV) is relatively constant across a large range of channel geometries.

FIG. 3 provides a nanofluidic chip 100 with a single nanochannel 105 of constant cross-sectional area. The top portion of the drawing is a top view of the nanofluidic chip 100 showing inlet 101 (at left), microchannel 102, detection region (represented as a filled rectangle, 104) and outlet 103 (at right). The bottom portion of the drawing in the center is the detection region in a top view, zoomed. The bottom portion of the drawing on the left and right are side views of the cross section, zoomed. The left drawing shows the cross section at the inlet of the nanochannel, while the right drawing shows the cross section at the outlet of the nanochannel.

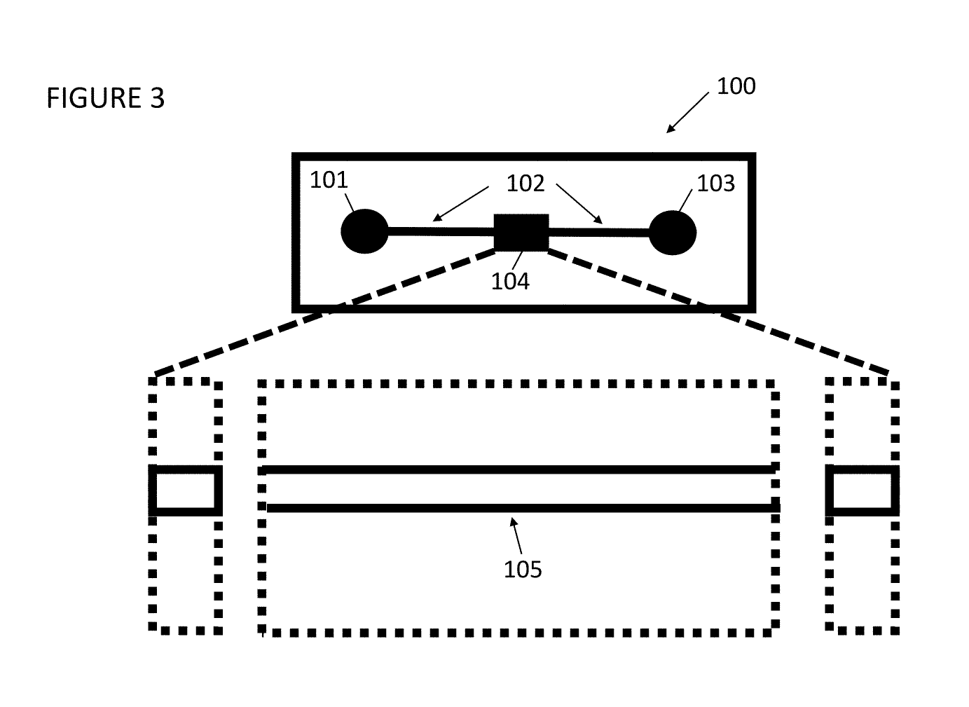

FIG. 4 provides a nanofluidic chip 200 with a single nanochannel 205 of variable cross section. The top portion of the drawing is a top view of the nanofluidic chip 200 showing inlet 201 (at left), microchannel 202, detection region (represented as a filled rectangle, 204) and outlet 203 (at right). The bottom portion of the drawing in the center is the detection region in a top view, zoomed. The bottom portion of the drawing on the left and right are side views of the cross-section, zoomed. The left drawing shows the cross section at the inlet of the nanochannel, while the right drawing shows the cross section at the outlet of the nanochannel.

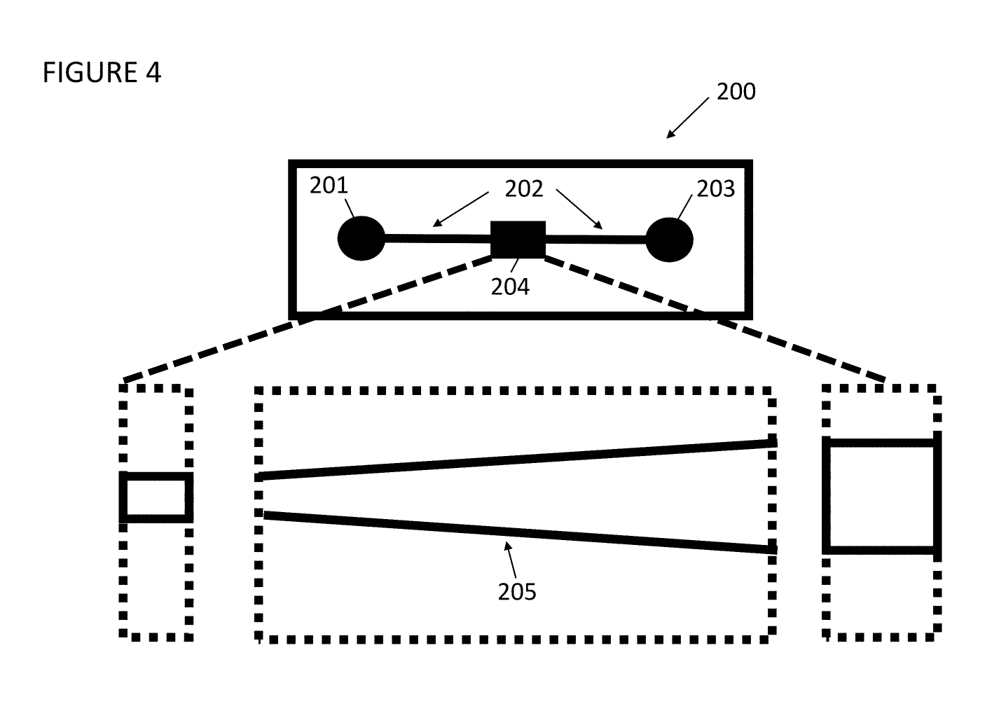

FIG. 5 shows a nanofluidic chip 300 with an array of nanochannels 305 of the same and of constant cross section. The top portion of the drawing is a top view of the nanofluidic chip 300 showing inlet 301 (at left), microchannel 302, detection region (represented as a filled rectangle, 304) and outlet 303 (at right). The bottom portion of the drawing in the center is the detection region in a top view, zoomed. The bottom portion of the drawing on the left and right are side views of the cross-section, zoomed. The left drawing shows the cross sections at the inlet of the nanochannels, while the right drawing shows the cross sections at the outlet of the nanochannels.

FIG. 6 provides a nanofluidic chip 400 with an array of nanochannels 405 of different and of constant cross section. The top drawing is a top view of the nanofluidic chip showing inlet 401 (at left), microchannel 402, detection region (represented as a filled rectangle, 404) and outlet 403 (at right). The bottom portion of the drawing in the center is the detection region in a top view, zoomed. The bottom portion of the drawing on the left and right are side views of the cross-section, zoomed. The left drawing shows the cross sections at the inlet of the nanochannels, while the right drawing shows the cross sections at the outlet of the nanochannels.

### DESCRIPTION OF THE EMBODIMENTS

### I. Definitions

By “analyte,” we mean any species to be detected. This may include, but is not limited to, nucleic acids (including DNA, RNA, and others), polymer, beads, biologics attached to beads, molecule capable of optical or electrical detection, molecule capable of binding to detectable molecules for optical or electrical detection, protein, cell, etc. The analyte(s) in a sample may be a species of interest (e.g., DNA to be sequenced) and/or an interferent (e.g., contaminant).

By “chip,” we mean an apparatus in planar form that can perform operations or assays on molecules. A chip may comprise one or more devices. A chip may be a fluidic chip or it may be a nanofluidic chip.

By “device,” we mean a component of a chip that can accommodate a sample and interface with an instrument, which, together, provide an experimental result for the sample. The device may include an inlet, an outlet, a fluid transport region comprising at least a detection region (e.g., nanochannels), and a means for interfacing with the detection system (such as any transparent layer so that a dye can be excited and emission can be received, including, but not limited to a transparent plastic layer that allows an intercalating dye to be excited and emission received). Devices may or may not be fluidically distinct from the inlet region to the detection region.

By “detection region,” we mean a region of a chip or device in which detection occurs.

By “detection system,” we mean any apparatus or system of apparatus capable of detecting an analyte by any means known in the art, including, but not limited to, optical methods (e.g., transparent window through which light from a radiation source (laser, mercury arc lamp, light emitting diode, etc.), transmitted light, fluorescence, luminescence, phosphorescence, etc. can pass; optics for directing incident, transmitted, and/or emitted light such as optical lens, mirrors, gratings prisms, and monochromators for colorimetric analysis; and detectors such as a camera, APD (avalanche photo diode) detector, and a PMT (photomultiplier tube) detector) and electrical methods (e.g., electrical contacts (such as electrodes or electrical sensors) and circuits for detecting a voltage change, current change, conductivity change, capacitive change, etc. due to the presence of analyte(s)).

The term “duty cycle” is used herein to describe detection of molecules (i.e., data points) in a given time, expressed as the number of molecules detected per unit time (e.g., minute) (i.e., throughput). If a molecule oscillates back and forth through the detection region so that it passes the detector more than one time, it would count as a single molecule detected in the duty cycle calculation even though it would be read by the detector multiple times.

By “instrument,” we mean machinery that integrates with a chip (and/or device), customer and software. The instrument may comprise user inputs, detection capability, and reporting capability.

By “macrochannel,” we mean a fluidic channel with the smallest dimension—whether cross-sectional width, depth, wall-to-wall distance, and/or diameter—that is at least 10 mm, for at least a portion of the length of the channel.

By “millichannel,” we mean a fluidic channel with the smallest dimension—whether cross-sectional width, depth, wall-to-wall distance, and/or diameter—that is at least 1 mm, but less than 10 mm, for at least a portion of the length of the channel.

By “microchannel,” we mean a fluidic channel with the smallest dimension—whether cross-sectional width, depth, wall-to-wall distance, and/or diameter—that is at least 1 μm to less than 1000 μm (or 1 mm), for at least a portion of the length of the channel.

By “nanofluidic,” we mean a fluidic channel with the smallest dimension—whether cross-sectional width, depth, wall-to-wall distance, and/or diameter—that is at least 1 nm to less than 1000 nm (or 1 μm), for at least a portion of the length of the channel. The prefix nano (as in nanochannel, etc.) also imparts the same meaning. For example, in some embodiments, a nanochannel may be ≤0.999, 0.99, 0.95, or 0.9 micrometer for at least a portion of the length of the channel.

By “nanofluidic chip,” we mean an apparatus in planar form having at least one device comprising at least one nanochannel that can perform operations or assays on molecules. A nanofluidic chip may comprise one or more devices.

By “sample” we mean a sample, such as, but not limited to a biological sample, for detection of a single analyte or multiple analytes. The multiple analytes may overlap in size distribution or not. The analyte(s) in a sample may be a species of interest (e.g., DNA to be sequenced) and/or an interferent (e.g., contaminant).

### II. Fluidic Chips

Optimizing the duty cycle for detection of a macromolecule on a fluidic chip can minimize assay run time, while maximizing the quality of the data obtained from an experiment (for example, by not having multiple DNA molecules travel through the detection area at the same time). Macromolecule detection functions most effectively when the peaks from different macromolecules are not confounded with each other during detection (e.g., each PMT data capture, which is defined by the sampling rate, reflects a voltage indicative of zero or one macromolecule, but not more than one macromolecule). This can, sometimes, require a longer duty cycle. However, users of fluidics devices are mindful of the time that these experiments can take and users also wish to optimize the duty cycle (i.e., shorten the duty cycle) so as to lessen the time a detection process takes. Finding the balance between the length of a duty cycle to obtain high-quality results and a short assay that streamlines a user's workflow calls for new methods and fluidic chips that can operate to optimize this duty cycle. Fine-tuning the cross-sectional area of fluid transport channels used for detection is a new method for controlling duty cycle. In a fluid transport channel with a smaller cross-section, the macromolecules may be more separated from each other than in a fluid transport channel with a larger cross-section.

The desired duty cycle varies with the concentration of the analyte for detection (whether known or unknown), the type of analyte being detected, and the type and quality of data desired by the user. The desired duty cycle may range from 1 molecule per minute to 1000 molecules per minute per fluid transport channel, for example 2 molecules per minute per fluid transport channel to 500 molecules per minute per fluid transport channel, or 2 molecules per minute per fluid transport channel to 100 molecules per minute per fluid transport channel. The devices and methods herein can adjust to a wide dynamic range of inputs depending on a plurality of features of the sample, including analyte concentration in a sample, and the features of the output desired by the user (from speed to precision). Some users may prefer 2 molecules per minute per fluid transport channel, 10 molecules per minute per fluid transport channel, or 50 molecules per minute per fluid transport channel. Other features may also affect the desired duty cycle. For example, higher fidelity detection systems can accommodate higher duty cycles, sometimes even more than 1000 molecules per minute per fluid transport channel. Lower fidelity detection systems may require lower duty cycles. Smaller nanochannels could accommodate higher duty cycles. Smaller macromolecules, such as shorter DNA, would also allow a higher duty cycle. If a user desired detecting multiple signals, such as multiple colors of fluorophores, a lower duty cycle might be appropriate.

In some embodiments, one way to optimize detection may be to ensure a sufficiently high fluid volume per unit time that passes through the detection region, which increases the number of macromolecules being detected. In order to separate data points, one could taper the cross section to spread out the molecules or add multiplex capability. Thus, one way to shorten run time is to run the macromolecules faster through the fluid transport channels (e.g., higher velocity). In this case, the volume of fluid that is transported through the fluid transport channels increases, so the number of macromolecules sampled increases. Similarly, one could have parallel channels to transport a higher volume of fluid in a given amount of time, increasing the number of macromolecules sampled.

Another way to increase volume of fluid is to make the cross section of the fluid transport channel bigger. If a device has two parallel fluid transport channels that experience the same electric field, the fluid transport channel that has a larger cross-sectional area will have a higher volume of fluid transported per unit time, increasing the number of macromolecules sampled.

A single nanochannel that is tapered (i.e., has a cross-sectional area that varies along its length) will have the same volume of fluid transport per unit time regardless of position along the length of the nanochannel. Because fluid is incompressible, that means the flow rate will have to be faster where the cross-section is smaller. Using a tapered channel and a detection window that can be positioned at various positions along the channel length can, thus, be used to optimize the separation between macromolecules, either with or without also tuning the volumetric flow rate (i.e., volumetric flow rate may be constant while the linear flow rate varies, or both may vary).

In some embodiments a fluidic chip for detecting an analyte comprises (a) at least one device comprising (i) a fluid inlet and (ii) at least one fluid transport channel that passes through a detection region, wherein (1) the at least one fluid transport channel has varying cross-sectional area as a function of position along the channel length and/or (2) the device comprises two or more fluid transport channels configured with two or more different cross-sectional areas; and (b) at least one fluid outlet, wherein the device provides for transport of single molecules across the detection region at a desired duty cycle by (1) allowing for detection of an analyte at a different position along the channel length based on the desired cross-sectional area and/or (2) allowing for detection of an analyte in a specific channel configured with the desired cross-sectional area.

This disclosure describes a variety of ways of controlling the duty cycle, either used alone or in combination. In some embodiments, the cross-sectional area within a fluid transport channel varies as a function of position along the channel length. Thus, in some embodiments, the two or more different cross-sectional areas are produced by having varying cross-sectional areas as a function of position along the channel length.

In some embodiments, the cross-sectional area within a fluidic transport channel is constant in at least some or all fluid transport channels. In these embodiments, the two or more different cross-sectional areas are produced by having different cross-sectional areas in different fluidic transport channels. The embodiments may also be combined such that different cross-sectional areas are produced by a combination of having varying areas as a function of position along the channel length and varying areas between different fluidic transport channels.

Having varying cross-sectional areas allows the user to optimize the duty cycle on a fluidic chip for a given experiment or given macromolecule to provide the most optimum environment for detection of the macromolecule.

The fluidic chip may comprise various types of fluid transport channels. It may comprise macrochannels, millichannels, microchannels, and/or nanochannels.

In some embodiments, a nanofluidic device capable of accommodating a range of analyte concentrations comprises (a) two or more nanochannels, fluidically connected at an inlet, (b) a throughput that differs within and/or between the at least two nanochannels due to different cross-sectional areas; and (c) a detection region. In some embodiments, at least two nanofluidic devices are comprised on a nanofluidic chip.

In some embodiments the fluid transport channels flow into an outlet shared by more than one fluid transport channel. Thus, the fluid transport channels (such as nanochannels) may be fluidically connected at the outlet. In some embodiments, the interface between a common outlet and each of the fluidically-connected fluid transport channels comprises a nanoscale orifice. The nanoscale orifice or nanochannel functions as an entropically-driven valve for macromolecules. In other words, the nanoscale orifice may limit macromolecular entry in the absence of a driving force (e.g., a favorable electrical voltage or fluidic pressure gradient).

A. Additional Aspects of Fluidic Chips

Fluidic chips are configured to accept sample and allow transport of the sample through fluidic channels to a detection region.

Sample inlet: A device may have means to accept fluid, such as vias, ports, capillaries and other reservoir and/or inlet design features, as is known in the art.

Sample: The sample may comprise a fluid with one or more of an analyte; a detection aide (e.g., intercalating dye, dye bound to a receptor that itself will bind to the analyte, beads, nanoparticles); a solution for preserving desired functional properties of the analyte and/or detection aide (e.g., buffered solutions of particular pH, solutions of particular osmolarity, solutions that do not denature, etc.); a preservation aide; transport aides, such as electroosmotic flow (EOF) inhibitors (e.g., PVP (polyvinylpyrrolidone) or PEG (polyethylene glycol)); detergents; other chemicals to (a) minimize biological growth, contamination, or increase in an interferent (e.g., EDTA (ethylene diamine tetraacetic acid); (b) protect the analyte from degradation (e.g., an antioxidant like DTT (dithiothreitol), ascorbic acid, DMSO (dimethyl sulfoxide), BME (beta-mercaptoethanol), which protects DNA from photoinduced cleavage), (c) to protect the detection aide from degradation; interferents, contaminants; one or more types of markers for calibration, quality control, etc.

Transport of the sample may be achieved via fluidic features, such as inlets, macrochannels, and/or channels with length and/or width that is <10 centimeter and/or <1 micrometer.

There may or may not be means to separate, purify, process, and/or concentrate the analyte of the sample after introduction to the device. For example, a device could comprise side nanochannels capable of accepting fluid, but not long DNA under a given voltage, thus concentrating the sample.

In one embodiment, there may be means to mix the detection aide with the analyte either before or after introduction to the device. For example, the detection aide may be added to the fluid before introduction to the device or the detection aide may be in dried format in the chip and dissolved by fluid entering into the chip (with optional mixing).

Multiple (e.g., parallel) fluidic channels of different widths and/or depths (e.g., originating from the same device) may be used. In this embodiment, difference in throughput from nanochannel to nanochannel is roughly a function of the difference in cross-sectional area.

Channels may be of length, width, and/or height that is macro/milli/micro/nano. The degree to which throughput may be varied independently of DNA velocity may be a function of channel cross section, such that the variables may be more independent in nanochannels than macrochannels. Channels may be interrogated simultaneously, serially, and/or selectively some but not all.

A fluid transport channel (or channels) may have tapering depth and/or width as a function of position along the channel and the ability to position the detection region at the optimal location along the fluid transport channel length. For example, DNA molecules that have confounded signals at one position along the fluid transport channel may be distinct at a position of decreased cross section along the same channel. Thus, channels could taper along their entire length or just a portion of the length. Channels could widen and/or narrow. Channels may be interrogated simultaneously, serially, and/or selectively some but not all.

B. Materials, Composition, and Fabrication of the Nanofluidic Chip

Chip material may be comprised of any material as is known in the art. For example, one or more of plastic; glass or fused-silica; silicon; silicone or rubber; adhesive or pressure sensitive adhesive; conductive material such as electrodes (e.g., carbon, metal); and coatings. In some embodiments, the nanofluidic chip is made of plastic. In some embodiments, a portion of the nanofluidic chip is made of injection-molded plastic.

Channels may be fabricated via any fabrication method known in the art. In one embodiment, a patterned layer may be bonded to a second layer. The second layer may be patterned or flat. Additional layers may be added, if desired or required, to add functionality, parallelize, etc.

Patterning of nanochannels may be accomplished via any method as is known in the art. In one embodiment, chips and devices are nano-patterned directly by a nanofabrication technique: for example, one or more of etching, photolithography, x-ray lithography, dip pen lithography, micromolding in capillaries (MIMIC), microtransfer molding, electron beam lithography, dry etching, wet etching, thermal scanning probe lithography, laser etching, high precision milling, electron discharge machining (EDM), focused ion beam (FIB) milling, nanoimprint lithography, etc. In another embodiment, a master may be patterned by a nanofabrication technique, and the master may be used to mold the nanopatterned chips or devices. In yet another embodiment, the master may be patterned by a nanofabrication technique, and then the master may be used as a mold to directly or indirectly pattern a tool for injection molding. Other molding techniques may be used alternatively or additionally, such as hot embossing, as is known in the art. In some embodiments, the wells are created using boss features.

In one embodiment, the master or mold may be fashioned with a draft angle to facilitate separation of the chip from the mold or master, or the mold itself from the master.

Bonding may be performed by any method as is known in the art. For example, one or more of pressure sensitive adhesive or tape, solvent assisted bonding, adhesive, plasma treatment or surface modification, conformal contact, laser welding, ultrasonic bonding, thermal bonding approaches, anodic bonding, induction welding, and clamping. In some embodiments, the bottom side of the nanofluidic chip is sealed using a solvent-bonding process.

In one embodiment, the surfaces may be modified to facilitate the assay, to perform the assay, or for other reasons. Modification may comprise plasma treatment, corona treatment, ozone or UV treatment, wet treatment (e.g., potassium hydroxide solutions), vapor polishing, or vapor deposition.

In one embodiment, additives may be on the chip in wet and/or dry form. For example, the chip may comprise: biochemical buffer or assay components, coatings, reagent, preservative, lysis components, dyes, etc.

In some embodiments, the nanochannel walls have a tapered profile constituting a draft angle. In some embodiments, a method of producing the nanofluidic chips described herein comprise producing a plastic nanofluidic chip using injection molding and fabricating the nanochannels with focused ion beam (FIB) milling, creating a draft angle. Thus, in some embodiments, a method of producing the nanofluidic chips described herein results in nanochannel walls having a tapered profile constituting a draft angle. In some embodiments, the draft angle is 0.5, 1, 1.5, 2, 2.5, 3, 3.5, 4, 4.5, 5, 5.5, 6, 6.5, 7, 7.5, 8, 10, 15, 20, 25, 30, 35, 40, or 45) degrees (°).

C. Detection System

The fluidic chips work in partnership with a detection system. The detection system may be any as is known in the art. For example, one or more of: colorimetric optical; fluorescence, phosphorescence, luminescence, etc.; and electrical (voltage, impedance, current, capacitance). A transparent window may be used to facilitate fluorescence, colorimetric, luminescence, phosphorescence, etc. Electrical methods may further include electrical contacts facilitating a voltage change, current change, conductivity change, capacitive change, etc.

In some embodiments, the method employs a detection system that does not move with respect to the chip during the detection step. In some embodiments, the method employs a detection system that does move or scan during the detection step. In some embodiments, analyte is combined with a detection aide either before or after introduction into the device. Again, detection aides include, but are not limited to, intercalating dye, dye bound to a receptor that itself will bind to the analyte, beads, nanoparticles.

The detection region may comprise (a) one or more of a two-dimensional region that aligns analyte in device to a detector, (b) fluidic channels (macro/milli/micro/nano dimensioned in width and or depth), (c) two or more fluidic channels (e.g., nanochannels), (d) fluidic channels from a single device or multiple devices, in which the multiple devices may have different samples. The fluidic channels may or may not be fluidically connected at the outlet. The fluidic channels may or may not be fluidically connected at the inlet.

Design factors for the detection region may comprise: a detection zone configured to align and interface with the working distance of an optical lens, a laser, a camera, APD (avalanche photo diode) detector, and/or a PMT (photomultiplier tube) detector, minimal background fluorescence, and a means for connecting electrical contacts to the instrument. The detector may operate to detect analyte from one or more than one nanochannels simultaneously, where the nanochannels may be from a single device or multiple devices.

Assay optimization to optimize and stabilize both signal and background or drift may employ methods as is known in the art.

D. Methods of Detecting an Analyte

The optimized duty cycle also allows a user to fine-tune a detection method based on factors particular to each experiment, for example varying concentrations of various macromolecules being detected or an unknown concentration of the macromolecule(s) being detected. This prevents unnecessary repeating of runs when the duty cycle was not optimized and the peaks from different macromolecules confound each other and/or are not optimally separated. It also prevents an unnecessarily slow duty cycle that does not optimize the user or researcher's time investment.

Thus, a method of detecting an analyte using a fluidic chip comprises (a) providing any of the fluidic chips described herein wherein (1) if the at least one fluid transport channel has varying cross-sectional area as a function of position along the channel length, detecting an analyte at a different position along the channel length based on the desired cross-sectional area in order to optimize duty cycle, and/or (2) if the device comprises two or more channels configured with two or more different cross-sectional areas, and (b) detecting an analyte in a specific channel configured with the desired cross-sectional area in order to optimize duty cycle.

In some embodiments, a method of detecting an analyte using a fluidic chip comprises: (a) providing a fluidic chip comprising (i) at least one device comprising a fluid inlet and at least one fluid transport channel that passes through a detection region and (2) a fluid outlet; and (b) adjusting the rate of transport of single molecules through the fluid transport channel across at least the detection region by: (i) adjusting the actuation energy applied to the analyte; electro-osmotic force tuning; and (iii) tuning of ionic strength and/or mobility, wherein adjusting the rate of transport of single molecules yields a desired duty cycle.

Electro-osmotic force comes from an ionic double layer that forms on the fluid transport channel walls. It can serve to complement or counteract flow rates when electrophoresis is used as the actuation energy. Increasing the concentration of an electro-osmotic force inhibitor (such as PVP) can increase or decrease the net mobility of the macromolecule through the fluid transport channel during electrophoresis, depending on whether the electro-osmotic force opposes or augments the electrophoretic force on the macromolecule. Ionic strength can also change velocity of the macromolecule through the fluid transport channel. Increasing ionic strength can slow the mobility of the macromolecule through the fluid transport channel during electrophoresis, due to charge shielding.

In some embodiments, when the cross-sectional area changes within a fluid transport channel, the fluid transport channel widens at or approaching the detection region. In some embodiments, when the cross-sectional area changes within a fluid transport channel, the fluid transport channel narrows at or approaching the detection region.

In one embodiment, the concentration of macromolecules (e.g., DNA macromolecules) may vary from experiment to experiment, and/or the actual concentration may be unknown or only approximately known. In these scenarios, there is a need for methods to optimize the duty cycle.

In one example to optimize the duty cycle, a nanofluidic device may have multiple parallel fluid transport channels in an array, each with a different cross-sectional area, which all originate from the same inlet, such that they all will transport molecules from the same fluid. The device may receive and transport a sample with an unknown concentration through the fluid transport channels of the detection region. The device may be optically transparent, allowing for excitation of intercalating dye that is bound to DNA, and then transmission of intercalating dye signal emission to a detector. The data may be post-processed by an algorithm, determining which nanochannels transported DNA with sufficient temporal spacing, such that data points are not confounded. Then, data may be aggregated from all such nanochannels. An alternative determination algorithm may only consider data from a subset of the nanochannels for which data points are not confounded, such as only those with a sufficiently high throughput. Thus, in this approach of utilizing multiple cross-sectional areas in parallel, a wide range of sample concentrations may be assayed with sufficient data throughput but not confounded data points. In the example of voltage driven sample flow through fluid transport channels of equivalent length, the average DNA temporal spacing along the flow direction in nano or microfluidic channels is proportional to 1/(cross-sectional area*DNA concentration). In other words, if width and depth both increase tenfold, the throughput will increase one hundredfold.

In both electro-kinetic flow and hydrodynamic flow conditions, for an identical driving force, DNA temporal spacing in channels with 100 nm×100 nm cross-section is larger than in 1 μm×1 μm channels. For a preferred DNA spacing, a 1 μm×1 μm channel can read DNA at a many-fold lower concentration at the same throughput as 100 nm×100 nm channels. If a device incorporates several channels with different cross-sectional areas, samples with a larger dynamic range of DNA concentrations may be read out at preferred DNA spacings.

In at least some embodiments, if the at least one fluid transport channel has varying cross-sectional area as a function of position along the channel length, the cross-sectional area is smaller closer to the inlet and the cross-sectional area is larger closer to the outlet. In other embodiments, the cross-sectional area is larger closer to the inlet and smaller closer to the outlet. In at least some embodiments, if the at least one fluid transport channel has varying cross-sectional area as a function of position along the channel length, at least one fluid transport channel tapers along its entire length. In at least some embodiments, if the at least one fluid transport channel has varying cross-sectional area as a function of position along the channel length, the fluid transport channels taper along a portion of their length. The fluid transport channel may also taper along its length for the whole length of the detection region or only a portion of the detection region.

In one example, a fluidic channel may taper along its entire length such that width and depth are 200 nm×200 nm at the inlet and 2000 nm×2000 nm at the outlet of the 300 micron-long channel. A fluid of uncertain RNA concentration may be flowed through the device via a pressure difference from inlet to outlet achieved with a vacuum of 10 in Hg applied at the outlet. Upon detecting an initial portion of the sample, an instrument may process the initial data using an algorithm. The algorithm may determine that the throughput is low, lengthening a sample run to obtain sufficient data points to yield useful statistical confidence in the data set, such that the vacuum must be increased to 15 in Hg at the outlet. After increasing the vacuum, the throughput increases but an unacceptable proportion of RNA molecules pass through the detector in multiples, confounding the data. Therefore, the detection region may move towards the inlet of the channel such that the RNA molecules are spatially separated (and moving faster). Upon identifying the optimal location for the detection region and the optimal actuation energy, the sample run may proceed with an optimal duty cycle.

In one embodiment, the tapering of a nanochannel may have benefits in signal detection. For example, the widening of a channel may cause a macromolecule to decelerate, such that it compresses, which may serve to spatially concentrate intercalating dye molecules that are bound to the macromolecule. Spatial concentration may increase the maximum signal peak obtained during detection, which may have benefits in signal detection.

Fluidic chips may passively accommodate flexibility (for example, by using an array, array of different cross section) and/or they may actively respond to determinations of throughput (for example by transmitting different or changing magnitudes of actuation energy, such as a voltage change).

For a given magnitude of actuation energy, the spacing of individual DNA molecules in the detection channel may be inversely proportional to the product of cross-sectional area of the detection channel and the DNA concentration. Therefore, reducing/enlarging the channel cross section by 10× may enable a 100× lower/higher, respectively, number of macromolecules per unit time (i.e., volumetric flow rate) to run through the channel at the same speed (i.e., linear flow rate). Such a change then impacts whether molecules pass through the detection region at the same time or not (i.e., doublets)—a higher volumetric flow rate at a given linear flow rate means a greater likelihood of such doublets. One possible method to increase the dynamic range of a given device is to employ multiple parallel channels with different cross-sectional areas. This enables a larger sample concentration range to be used with the device. An alternative or additional method to increase dynamic range would be to use a channel that employs a gradual change in cross-sectional area (increasing or decreasing) from the entrance to the exit. This could also be a set of parallel channels with either the same gradual change in channel cross section or an array of channels with different cross-sectional profile changes. The detection point, for example, a laser spot, could be dynamically moved along the channel(s) to find the optimal DNA spacing to ensure molecules are not too far apart or so close that they overlap. A third way is to use hydrodynamic flow focusing, as discussed further in the following actuation energy section, to focus DNA to a smaller cross-sectional area spacing the DNA molecules further apart. These methods for spacing individual DNA molecules could be operated in a hydrodynamic or electrophoretic manner. A fourth way to increase dynamic range and/or optimize duty cycle is to use more than one fluidic channel with the same dimensions, such as an array of parallel channels. A fifth way is to change the actuation energy method or magnitude itself.

1. Actuation Energy

The fluidic chip operates using an actuation energy to move a macromolecule through the fluid transport channels and past the detection region. The actuation energy comprises voltage, current, hydrostatic pressure, pneumatic pressure, vacuum pressure, flow focusing, or centrifugal force. Thus, fluid transport may be achieved by one or more of: electrophoresis (voltage or current drop), pressure-driven flow (hydrostatic, positive pressure, vacuum, centrifugal), or capillary forces.

Thus, the fluidic chip and more methods operate to provide transport of single molecules across the detection region by: (a) electrodes capable of applying a voltage difference, wherein the electrodes are in contact with liquid in or on the chip; (b) pneumatic adapters to apply pressure or vacuum integrated with a chip; and/or centrifugal forces.

Thus, transport of single molecules may be achieved in one of a variety of ways: (a) electrodes that apply a voltage or current difference may be integrated by placing in contact with liquid on/in the chip; (b) pneumatic adapters to apply pressure or vacuum may be integrated with a chip (e.g., adapters on fluid reservoirs of a chip); (c) rotation of the chip and optionally other system components to generate centrifugal forces may be applied, etc.

In flow focusing, the sample fluid flow (i.e., the fluid comprising the macromolecule for detection) is constrained and accelerated by parallel fluid streams flowing alongside of it. The cross-sectional area of sample fluid flow may be controlled by changing the flow rate of the buffer solutions around it, such that increasing the rate of flow of the buffer would shrink the cross-sectional area in a flow focusing embodiment.

Hydrodynamic flow focusing is an approach to control the cross-sectional position, speed, mixing, and other attributes of one or more fluids flowing in parallel in a channel. For example, fluid A may comprise buffer and macromolecule to be assayed, while fluid B may comprise buffer but no macromolecule. Fluid B may serve as a “sheath fluid”. If fluid A and B flow parallel to each other in a laminar flow profile within the same channel, each will consume a proportion of the cross-sectional area, and there may be a degree of mixing due to diffusion. Increasing the volumetric flow rate of fluid A while maintaining the volumetric flow rate of fluid B may increase the proportion of the total cross-sectional area consumed by fluid A while decreasing the proportion consumed by fluid B. To maintain the same volumetric flow rate, fluid B may then flow faster, which may separate macromolecules that comprise fluid B. This may be a method of tuning the duty cycle by changing the speed of the macromolecule-containing fluid, whether or not the volumetric flow rate changes, such that macromolecules separate as they pass through the detection region.

2. Opportunities to Adjust Duty Cycle

The duty cycle may be adjusted at any of multiple time points. Adjusting the duty cycle includes adjusting the rate of transport of macromolecules through the detection region and/or adjusting the spacing between macromolecules through the detection region. In some embodiments, the spacing of molecules is appropriate for the user's requirements, but the transport rate could be increased to shorten the duration of an experiment without reducing signal quality. In other embodiments, the spacing may not yet be adequate (either too wide and resulting in inefficiency or too narrow and increasing the risk of confounding signals).

The duty cycle may be adjusted in order to fine tune between transport rate and molecule spacing. There are several design decisions regarding devices, systems and/or methods that impact transport rate and/or molecule spacing. To impact transport rate, one may increase the average cross-sectional area of a particular channel, add multiple channels in parallel, reduce the length of one or more channels, increase the magnitude of the actuation energy (e.g., voltage or fluid pressure difference, etc.), etc. To increase molecule spacing, one may lower volumetric flow rate via any means or approach as is known in the art, introduce a channel of variable cross-sectional area and detect at a portion of the channel that is narrower, etc.

For example, in some embodiments, the adjustments to the duty cycle are based on pre-run factors. For example, during the sample preparation, enzyme or buffer components may be selected to improve run throughput. In some embodiments, the adjustments to the duty cycle are based on closed loop feedback during an experimental run. In some embodiments, adjustments to the duty cycle are based on post-run analysis. In some embodiments, the post-run analysis leads to culling a portion of the dataset. In some embodiments, the experimental run is conducted under multiple conditions and post-run one or more of those conditions is selected based on optimization of duty cycle.

In some embodiments, during the course of a sample run, initial data may be used as real-time feedback to improve data collection for the rest of the sample run. For example, data collection throughput or quality that is partially collected from a sample run may provide feedback for adjustments, including, but not limited to, cutting a run short or making a change to improve data quality. For example, in one embodiment, when the DNA (also including other forms of nucleic acids) to be evaluated by the method described herein has a known reference sequence, reads of DNA from an initial run may be randomly sampled for aligning with the reference sequence to get an indication of quality and provide real-time feedback on optimization of the duty cycle for the rest of the run.

In some embodiments, duty cycle optimization may be used to improve the quality of restriction site mapping of DNA (for example, as described in WO 2014/164739; U.S. Pat. Nos. 9,255,288; 9,970,898; 9,618,479; 10,106,848, the content of all of which are incorporated by reference herein for the purpose of description of DNA mapping applications). If the DNA to be evaluated by the method described herein has a known reference sequence, reads of DNA may be randomly sampled for an initial run for aligning with the reference sequence to investigate an abundance of missed cuts or whether the distribution of the number of fragments fits what is expected for the mapping conditions (genome, molecule size and restriction enzyme) and provide real-time feedback on optimization of the duty cycle for the rest of the run.

In some methods, conditions are selected based on optimization of duty cycle and those conditions are implemented using a different device in a different experimental run.

The adjustments to the duty cycle may be based on different factors. For example, they may be based on the density of signal spikes (also known as signal bursts, signal pulses, signal peaks, or transient signals) above a threshold; time averaged signal; likelihood of adjacent spikes confounding each other in a given run time; and/or ability to identify and cull individual confounding spikes from a dataset. These adjustments can enhance the quality of detection processes.

The voltage applied to the macromolecule passing through each fluid transport channel may be the same. For example, the voltage applied to the single molecule may not change during an experimental run.

On the other hand, actuation energy may be adjusted to fine-tune the duty cycle. One or more of the following variables may be adjusted to optimize the duty cycle: applied voltage, applied current, applied fluid pressure, applied vacuum, applied centrifugal force.

In some embodiments, the macromolecule flows through the detection region only one time. In other embodiments, the single molecule flows through the detection region multiple times. If a macromolecule flows through the detection region multiple times it provides additional opportunities to adjust the duty cycle. This may be accomplished using oscillatory actuation. For example, oscillatory actuation may be achieved by periodically reversing polarity of the fluid transport channel.

For example, a device comprises 10 nanochannels of different width and/or depth that run in a parallel array. DNA of an unknown sample concentration and unknown populations flows through all 10 nanochannels at a pre-set speed. Detection occurs on all 10 nanochannels simultaneously. During post-run analysis, nanochannels for which DNA throughput is too high, leading to confounding signals, is discarded. Data that is clean is aggregated across the remaining nanochannels and analyzed to yield a sufficiently large dataset.

Inputs that may be used to determine a selected duty cycle comprise: customer input (e.g., maximum run time, desired minimum statistical confidence in results, selection of particular programs, # of devices on a single chip to pool data across, # of chips to pool data across, sequencing purpose or application, etc.); hardwired controls or algorithmic rules in an instrument; feedback from an initial test run, leading to closed-loop control of the subsequent experimental run (e.g., initial test run shows DNA duty cycle is 10 molecules per minute per fluid transport channel, which is below the threshold of adjacent molecules confounding, so the duty cycle is raised to 30 molecules per minute per fluid transport channel to reduce run time by 3×; and post-run culling of data from any fluid transport channels for which duty cycle is too high, such that results and conclusions are based on un-culled channel data.

Algorithms that incorporate inputs and select the duty cycle may comprise any as is known in the art. For example: process signal and compare spike density to threshold, time averaged signal, factor in likelihood of adjacent spikes confounding each other in a given run time, factor in ability to identify and cull individual confounding spikes from dataset.

Variables that may be tuned to optimize the duty cycle comprise one or more of a change in and/or adjustment of energy applied to a fluid and/or analyte (e.g., applied voltage, applied current, applied fluid pressure, applied vacuum, applied centrifugal force, etc.). This change may be based on feedback (closed loop) or may be pre-programmed irrespective of signal characteristics (open loop). For example, one open loop approach is to collect data serially at different applied voltages, then post-process and select the duty cycle for determining results and conclusions. This change could be a reversal of polarity as a molecule is detected, to enhance detection. Reversing polarity may slow the duty cycle while improving data quality. Variables that may be tuned to optimize the duty cycle comprise one or more of multiple (e.g., parallel) fluidic channels that are originating from the same device in an array, originating on different devices of the same chip, originating on different chips of the same run, and/or interrogated simultaneously, serially, and or selectively some but not all.

### III. Methods of Using the Fluidic Chips

The present nanofluidic chips may be used for a plurality of different assay types. Assays may be performed for size determination of an analyte, including average size and coefficient of variation (CV). Assays may also look quantitatively at a sample to determine the number of analyte molecules per volume or with respect to other populations of analyte or one or more markers. Assays may also provide other information related to the analyte, such as strain typing, identification of differences in sequence, or genetic mapping. In some embodiments, assay conditions (e.g., enzymatic activity, chemical equilibria, etc.) may be adjusted via the adjustments to the duty cycle based on closed loop feedback (e.g., electrophoretic voltage control). For example, DNA transport speed (via controlling electrophoretic voltage) can be tuned to obtain reliable digestion at maximum throughput while maintaining assay performance (i.e., full enzymatic cleavage of DNA before the resultant DNA fragments reach the detection region of the nanochannel). In another example, chemical equilibria between buffers that are introduced at either end of a nanochannel may be tuned and maintained by controlling electrophoretic voltage (e.g. the EDTA in the DNA sample at nanochannel entrance and the magnesium ions (Mg²⁺) in the buffer at nanochannel exit—lack of equilibrium can lead to pre-mature or incomplete enzymatic cleavage. After the Mg²⁺ and EDTA concentrations have been adjusted, electrophoretic voltage can be a secondary parameter that can fine-tune the balance.

As an analyte, DNA (also including other forms of nucleic acids) may be evaluated to determine its size and to provide quality control metrics after DNA sequencing. DNA may also be assessed through detection of agglomerates. Apoptosis assays may also be performed in cancer research.

Long molecules of DNA (also including other forms of nucleic acids) may also be evaluated and sized on the nanofluidic chips. This allows for strain typing. Pathogen identification may also be conducted through sequencing DNA fragments for which order is maintained, for example, to identify the exact pathogen causing an infection and avoid or reduce problems with antibiotic resistance. Pathogen identification may also be employed for sanitation reasons in medical facilities or in the food industry. In some embodiments, DNA may be collected from the nanofluidic outlet after sizing for post-processing or use.

DNA (and other forms of nucleic acids) may also yield additional information on the nanofluidic chips through a DNA/nucleic acid mapping process. For example, the quality of a mapping analysis may be due, in part, to the degree of coverage of the genome (e.g., 30× coverage), and optimizing the duty cycle may allow for an appropriate balance between degree of coverage and run time for a sample of known or unknown concentration. DNA/nucleic acid may be cleaved into fragments before sizing, yielding further information. Optimizing of duty cycle has special advantages in a mapping application because it allows the user to further separate the fragments.

Other forms of single-molecule detection may also occur on the nanofluidic chips including protein sizing with an amine binding dye (e.g., Alexa Fluor™ 488 Succinimidyl Ester, Invitrogen); RNA sizing or detection; digital assays with DNA, RNA, and/or other nucleic acids; DNA fingerprinting (for forensic, medical, pathogen, or GMO testing). Clogging of analytes (e.g., DNA) may impact throughput and data quality of assays performed on nanofluidic chips. In some embodiments, unwanted clog may be dislodged and eliminated (or reduced or mitigated) by adjusting applied voltages (e.g., by an electrophoretic voltage) and/or illuminating the clogs or the nanochannel with a high-intensity light source (e.g., LED or Sola light) to disrupt the clogs thermally or photolytically. Then, the dislodged DNA may be culled from the dataset by excluding or filtering any data collected during this cleaning cycle. In some embodiments, dislodging a clog of the analyte or the single molecule from the fluid transport channel is achieved by applying an electrophoretic voltage to the fluidic transport channel temporarily or by illuminating the fluid transport channel with a high-intensity light source.

In some embodiments, the adjustment of applied voltages includes slowly increasing the voltages. In some embodiments, the adjustment of applied voltages includes alternating very high voltage with no voltage. In some embodiments, the applied voltages may be applied as pulses. In some embodiments, the adjustment of applied voltages includes varying the voltages using a sinusoidal, square-wave, saw-tooth-wave or other waveform as will be appreciated in the art. In some embodiments, the adjustment of applied voltages includes reversing voltages.

In some embodiments, the illumination of the clogs or nanochannel with a light source includes illuminating the clogs or nanochannel with a high-intensity light (e.g., Sola light). In some embodiments, the illumination of the clogs or nanochannel with a high-intensity light includes sweeping a laser light over the clogs or the entire channels at relatively high power.

In some embodiments, a protocol for adjusting applied voltages may be run due to a stimulus (e.g., when a throughput rate is slowing or becoming inconsistent). In some embodiments, the adjustment protocol may be run periodically (e.g., every 15 min for 10 sec, then return to the data collection protocol).

Thus, the nanofluidic devices have significant utility in the art.

Fluid may be primed before the assay begins or during the assay. Chips may be pre-primed during production or primed by the customer at time of use.

Sample entry to the device may be achieved by any technique as is known in the art. For example, one or more of via or port, tubing interface, hole or orifice.

### EXAMPLES

### Example 1: Electrophoretic Assay for Sizing DNA, RNA, or Protein with Preset Duty Cycle Parameters

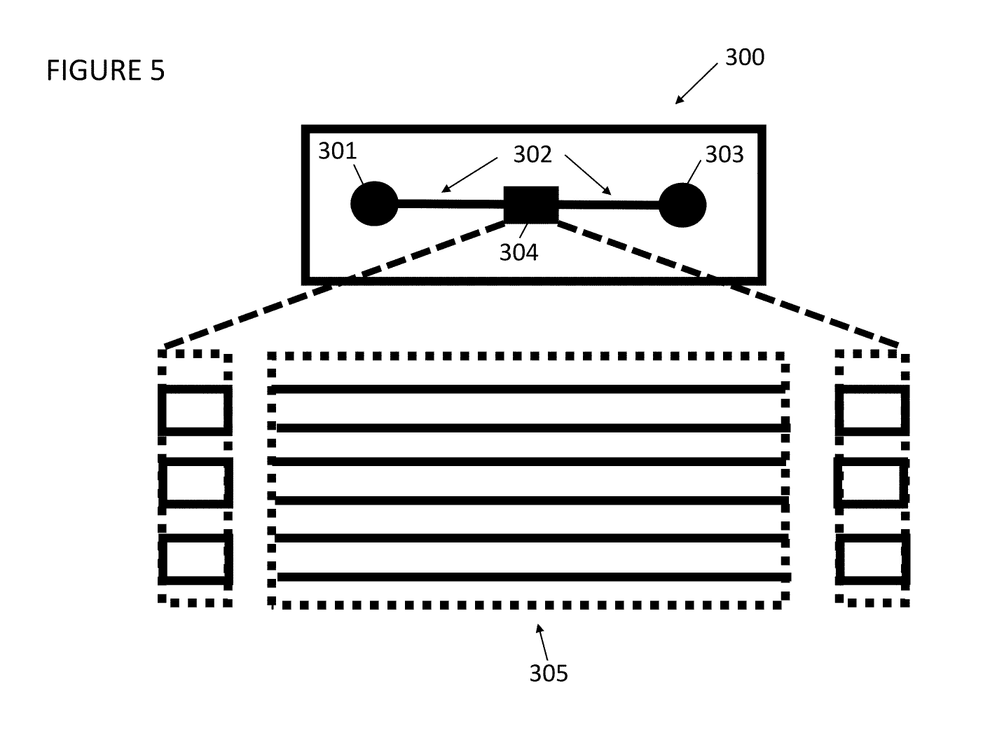

In one example, an electrophoretic assay for sizing DNA, RNA, or protein is employed to determine the size, precision and concentration of a sample that comprises DNA. The sample is placed into an inlet well of a nanofluidic chip according to FIG. 5 after the chip is primed with buffer solution to allow for electrophoretic transport. The nanofluidic chip is placed into an instrument comprising optics, a laser, PMT detector, camera, and processing and algorithm software. Electrodes that apply a voltage are placed into wells to transport DNA from a negatively-biased electrode to a positively-biased electrode, through a detection region comprising the nanochannels. DNA transports through the detection region. The algorithm detects each DNA molecule and determines that the molecules are spaced sparsely. The number of molecules that pass through the detection region is 3 molecules per minute (i.e., duty cycle). The algorithm is hardcoded that the optimal duty cycle is 50 molecules per minute. The applied voltage is then increased by 1600% from 10V to 160V, based on a hardcoded estimate for the chips and buffer system. The duty cycle is then measured for 1 minute and it has increased to 48 molecules per minute. The applied voltage is then increased another 10% to 176V, yielding a duty cycle of 50 molecules per minute. At the end of the run, all the data is aggregated regardless of duty cycle because within the range of voltage and resultant DNA transport speed experienced, precision and accuracy are not impacted. The increase in voltage decreases run time by ˜18× while yielding the desired statistical confidence in the sizing results.

### Example 2: Electrophoretic Assay for Sizing DNA, RNA, or Protein with Assay Optimizing Speed Over Precision

In a second example similar to Example 1 employing a chip according to FIG. 5, the customer pre-specifies in the software that the estimate of size may be rough. This customer has subjectively determined that precision is not needed and speed is preferable. Based on this input, the total number of data points acquired is reduced by 10× in addition to a ˜10× optimization of DNA speed, yielding a ˜100× reduction in assay run time.

### Example 3: Electrophoretic Assay for Sizing DNA, RNA, or Protein with Fluid Pressure Gradient Created by Applied Vacuum Pressure at the Outlets Used as Actuation Energy

In a third example similar to Example 1 employing a chip according to FIG. 5, a fluid pressure gradient, created by an applied vacuum pressure at the outlets, is used as the actuation energy (a driving force) instead of an applied voltage. Vacuum pressure is then increased to optimize the duty cycle and decrease run time.

### Example 4: Electrophoretic Assay for Sizing DNA, RNA, or Protein with Fluid Pressure Gradient Created by Applied Driving Fluid Pressure at the Inlets Used as Actuation Energy

In a fourth example similar to Example 1 employing a chip according to FIG. 5, a fluid pressure gradient, created by an applied driving fluid pressure at the inlets, is used as the actuation energy (a driving force) instead of an applied voltage. Applied driving fluid pressure is then increased to optimize the duty cycle and decrease run time.

### Example 5: A Genomic Mapping Assay on a Nanofluidic Chip

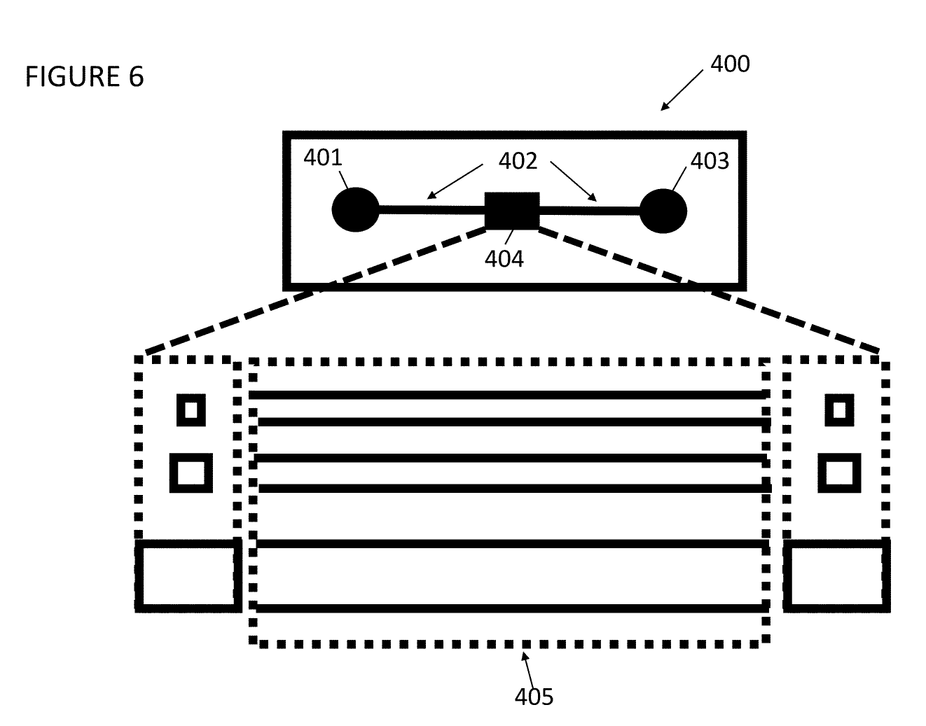

In a fifth example, a genomic mapping assay is run in a nanofluidic chip according to FIG. 6. In this assay, each DNA molecule is cleaved in a nanochannel by enzyme, the order of resultant fragments (i.e., the cleaved DNA molecule) is maintained during flow through the nanochannel, and then each fragments size is determined in a detection region.

Such assays can be long (24 hours) if mapping an entire human genome. To minimize assay time, there are parallel nanochannels, each of which is simultaneously addressable. The DNA sample has an unknown concentration.

There are 30 parallel nanochannels. The parallel nanochannels range in cross-sectional area but are all the same length. The first set of ten nanochannels have a constant width of ˜100 nm and a constant depth of ˜100 nm. The second set of ten nanochannels have a constant width of ˜300 nm and a constant depth of ˜300 nm. The third set of ten nanochannels have a constant width of ˜1000 nm and a constant depth of ˜1000 nm. The benefit in throughput at a constant driving voltage of the second set vs. the first set is 9×. The benefit in throughput at a constant driving voltage of the third set vs. the first set is 100×.

After 10 minutes of running, the duty cycle for each set of nanochannels is 0.01 DNA molecule per minute per nanochannel, 0.09 molecules per minute, and 1 molecule per minute per nanochannel, respectively. The optimal duty cycle is 2 molecules per minute per nanochannel for average size determination. However, the optimal duty cycle is 0.02 molecules per minute per nanochannel for an onboard quality control test that helps improve data quality. Therefore, the driving electrophoretic voltage is increased from 3V to 7V such that the third set of nanochannels increase to 2 molecules per minute per nanochannel, the second set to 0.18 molecules per minute per nanochannel, and the first to 0.02 molecules per minute per nanochannel. Data from all 30 nanochannels are aggregated to collect mapping data, with the third set of nanochannels disproportionately contributing more of the data. Data from the first set of nanochannels are used for the onboard QC test. The run time is ˜200× shorter than if all channels were the same size as the first set and no voltage feedback was applied.

### Example 6: A Genomic Mapping Assay on a Nanofluidic Chip while Reducing Unacceptable Number of Doublets

In an example similar to Example 5 employing a chip according to FIG. 6, a DNA sample with an unknown concentration is characterized in a mapping assay. Data is collected at a driving electrophoretic voltage of 10V. After the run is complete, the data is evaluated. Data from the third set of nanochannels has a duty cycle of 10 molecules per minute per nanochannel. Data from the second set of nanochannels has a duty cycle of 0.9 molecules per minute per nanochannel. Data from the first set of nanochannels has a duty cycle of 0.1 molecules per minute per nanochannel.

After the run is complete, an unacceptable percentage of doublets are observed in the third set of nanochannels such that data quality is poor. In contrast, the data quality is good for the second and first set of nanochannels, such that the data of these 20 nanochannels are aggregated and used. In this example, there is no pre-set optimal duty cycle—instead, the data for particular sets of nanochannels are characterized and then discarded during post-processing if poor.

### Example 7: A Genomic Mapping Assay on a Nanofluidic Chip Comprising an Array of 20 Parallel Nanochannels

In another example, DNA is sized in a device comprising an array of 20 parallel nanochannels, all of which have a length of 200 microns. The depth of the nanochannels is 1000 nm at their entrance, and then the floor tapers linearly such that the depth is 100 nm at the nanochannel exit. The width of the first set of 10 nanochannels is 100 nm across the entire length and the width of the second set of 10 nanochannels is 1000 nm across the entire length.

During the sizing run, both electrophoretic driving voltage and detection position along the length of the nanochannels is used to optimize the duty cycle. A wide range of throughput is possible. Increasing the driving voltage will increase duty cycle. Similarly, the second set of nanochannels has a larger cross section than the first, so the second set has a higher volumetric flow rate and a larger duty cycle per nanochannel. Positioning the laser and detector at different points along the length of the nanochannels will space apart or compress molecules spatially while also impacting speed. For instance, at the exit of the nanochannels, molecules will be moving faster than farther upstream due to the decreased depth. If there is a speed limitation, above which assay performance deteriorates, a position can be selected to optimize between speed and data quality.

### Example 8: Fragment Sizing with Nanochannels of Various Sizes

Fragment sizing was performed with various nanochannel widths of the device described herein to investigate the range of the width of the nanochannels over which the quality of fragment sizing data is not appreciably compromised. Nanochannels of greater width are generally easier to fabricate at lower cost and with higher device yield.

Testing was carried out by driving a DNA ladder with 200 bp, 1 kbp, 2 kbp, 3 kbp, 5 kbp, and 10 kbp DNA molecules through 4 devices which contained square cross-section nanochannels of various widths and depths (200 nm, 250 nm, 300 nm, 400 nm, 500 nm, 750 nm, and 1000 nm). The samples containing a DNA ladder with 200 bp, 1 kbp, 2 kbp, 3 kbp, 5 kbp, and 10 kbp were prepared in an electrophoresis buffer solution. The sample was placed into an inlet well of a nanofluidic chip according to FIG. 6 after the chip is primed with buffer solution to allow for electrophoretic transport. The nanofluidic chip was placed into an instrument comprising optics, a laser, PMT detector, camera, and processing and algorithm software. Electrodes that apply a voltage were placed into wells to transport DNA from a negative to a positive electrode, through a detection region of the nanochannels. PMT data was collected for 10 minutes from every channel.

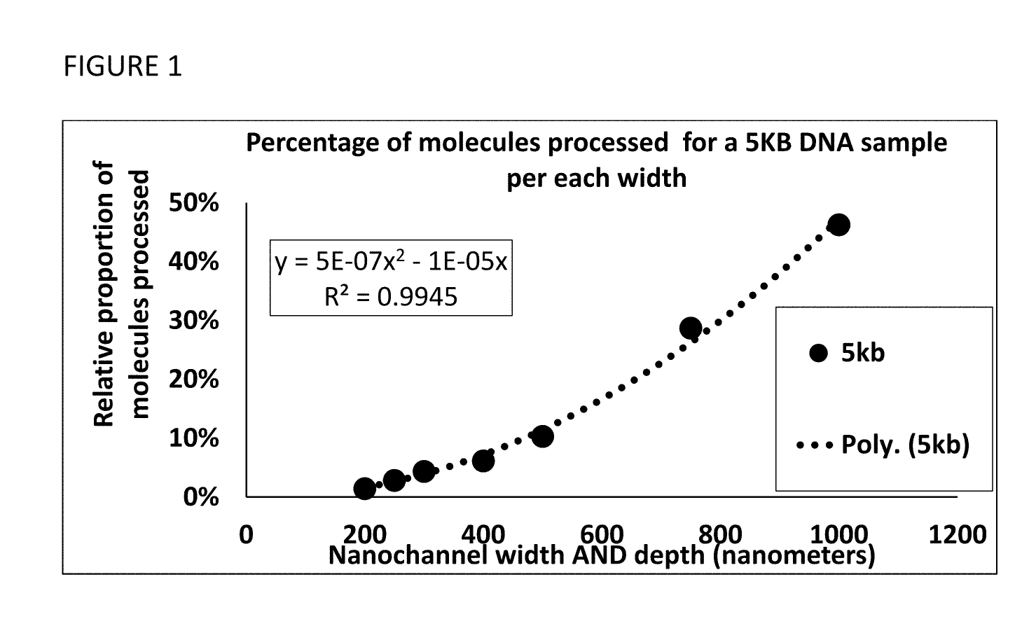

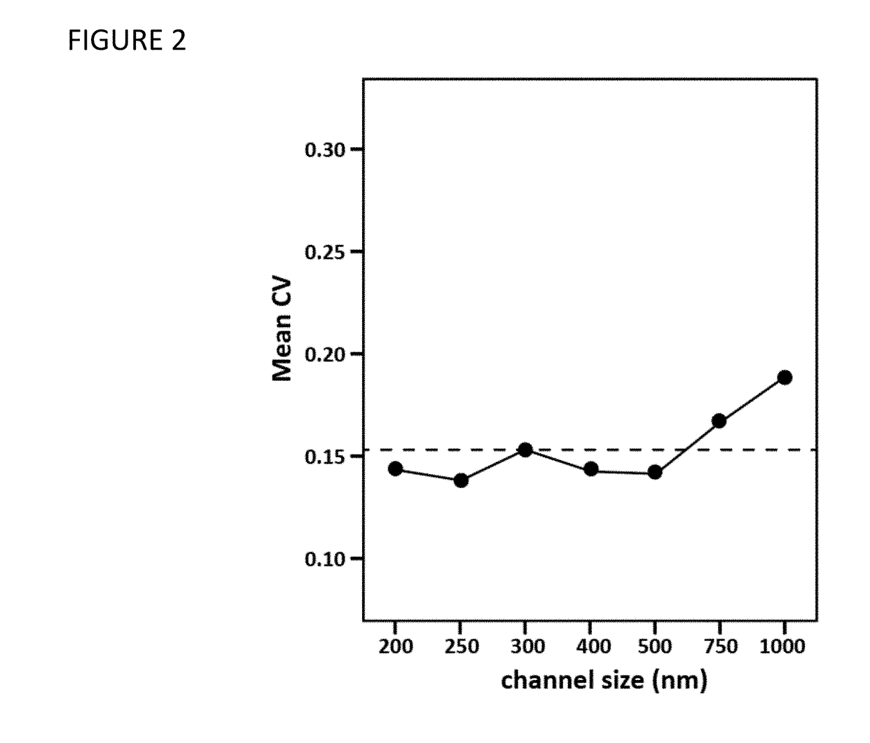

FIG. 1 shows the results from the run of a sample comprising 5 kbp sized DNA. Similar results are obtained for each of the ladder constituents (with 200 bp, 1 kbp, 2 kbp, 3 kbp, 5 kbp, and 10 kbp). Electrophoretic-based throughput through a nanochannel increased as nanochannel width and depth increased, at a rate of x², where x² is proportional to the cross-sectional area of the channel. There was approximately a 25-fold increase in the percentage of molecules passing through the 1000-nm wide by 1000-nm deep channel relative to the percentage of molecules passing through the 200-nm wide by 200-nm deep channels. As shown in FIG. 2, the relative coefficient of variation (CV) is relatively constant across a large range of channel sizes.

### Example 9: Certain Embodiments

Item 1. A fluidic chip for detecting an analyte comprising:
a. at least one device comprising
   i. a fluid inlet and
   ii. at least one fluid transport channel that passes through a detection region, wherein (1) the at least one fluid transport channel has varying cross-sectional area as a function of position along the channel length and/or (2) the device comprises two or more fluid transport channels configured with two or more different cross-sectional areas; and
b. at least one fluid outlet,
   wherein the device provides for transport of single molecules across the detection region at a desired duty cycle by (1) allowing for detection of an analyte at a different position along the channel length based on the desired cross-sectional area and/or (2) allowing for detection of an analyte in a specific channel configured with the desired cross-sectional area.

Item 2. The fluidic chip of item 1, wherein the duty cycle comprises a desired rate of detection.

Item 3. The fluidic chip of any one of items 1-2, wherein the duty cycle comprises a desired spacing between molecules.

Item 4. The fluidic chip of any one of items 1-3, wherein the cross-sectional area within at least one fluid transport channel varies as a function of position along the channel length.

Item 5. The fluidic chip of any one of items 1-4, wherein the cross-sectional area within at least one fluidic transport channel is constant.

Item 6. The fluidic chip of any one of items 1-5, wherein at least one fluid transport channel is a macrochannel.

Item 7. The fluidic chip of any one of items 1-6, wherein at least one fluid transport channel is a millichannel.

Item 8. The fluidic chip of any one of items 1-7, wherein at least one fluid transport channel is a microchannel.

Item 9. The fluidic chip of any one of items 1-8, wherein at least one fluid transport channel is a nanochannel.

Item 10. A method of detecting an analyte using a fluidic chip comprising:

providing the fluidic chip of any one of items 1-9;

wherein (1) if the at least one fluid transport channel has varying cross-sectional area as a function of position along the channel length, detecting an analyte at a different position along the channel length based on the desired cross-sectional area in order to optimize duty cycle, and/or (2) if the device comprises two or more channels configured with two or more different cross-sectional areas, detecting an analyte in a specific channel configured with the desired cross-sectional area in order to optimize duty cycle.

Item 11. A method of detecting an analyte using a fluidic chip comprising
a. providing a fluidic chip comprising (1) at least one device comprising a fluid inlet and at least one fluid transport channel that passes through a detection region and (2) a fluid outlet;
b. adjusting the rate of transport of single molecules through the fluid transport channel across at least the detection region by:
   i. adjusting the actuation energy applied to the analyte;
   ii. electro-osmotic force tuning;
   iii. tuning of ionic strength and/or mobility; or
   iv. a combination of two or more chosen from (i), (ii) and (iii),
      wherein adjusting the rate of transport of single molecules yields a desired duty cycle.

Item 12. The method of item 11, wherein the actuation energy is controlled by at least one chosen from voltage, current, pneumatic pressure, vacuum pressure, flow focusing, and/or centrifugal force.

Item 13. The device of any one of items 1-9 or the method of any one of items 10-12, wherein transport of single molecules across the detection region is achieved by:
a. electrodes capable of applying a voltage or current difference integrated with the chip, optionally the electrodes being placed in contact with liquid in or on the chip;
b. pneumatic adapters to apply pressure or vacuum integrated with the chip, optionally the adapters being on fluid reservoirs of the chip;
c. rotation of the chip and optionally other system components to generate centrifugal forces; or
d. a combination of two or more chosen from (a), (b) and (c).

Item 14. The device of any one of items 1-9 or 13 or the method of any one of items 10-13, wherein the adjustments to the duty cycle are based on pre-run factors.

Item 15. The device of any one of items 1-9 or 13-14 or the method of any one of items 10-14, wherein the adjustments to the duty cycle are based on closed loop feedback during an experimental run.

Item 16. The device of any one of items 1-9 or 13-15 or the method of any one of items 10-15, wherein the adjustments to the duty cycle is based on post-run analysis.

Item 17. The device of any one of items 1-9 or 13-16 or the method of any one of items 10-16, wherein the post-run analysis leads to culling a portion of the dataset.

Item 18. The device of any one of items 1-9 or 13-17 or the method of any one of items 10-17, wherein the experimental run is conducted under multiple conditions and post-run one or more of those conditions is selected based on optimization of duty cycle.

Item 19. The method of any one of items 10-18, wherein conditions are selected based on optimization of duty cycle and those conditions are implemented using a different device.

Item 20. The device of any one of items 1-9 or 13-18 or the method of any one of items 10-19, wherein the adjustment to the duty cycle is based on
a. spike density above threshold;
b. time averaged signal;
c. likelihood of adjacent spikes confounding each other in a given run time;
d. ability to identify and cull individual confounding spikes from a dataset; or;
e. a combination of two or more chosen from (a)-(d).

Item 21. The device of any one of items 1-9, 13-18, or 20 or the method of any one of items 10-20, wherein the analyte is of unknown concentration in a sample.

Item 22. The device of any one of items 1-9, 13-18, or 20-21 or the method of any one of items 10-21, wherein the fluid transport channel widens at or approaching the detection region.

Item 23. The device of any one of items 1-9, 13-18, or 20-22 or the method of any one of items 10-22, wherein the fluid transport channel narrows at or approaching the detection region.

Item 24. The device of any one of items 1-9, 13-18, or 20-23 or the method of any one of items 10-23, wherein if the at least one fluid transport channel has varying cross-sectional area as a function of position along the channel length, the cross-sectional area is smaller closer to the inlet and the cross-sectional area is larger closer to the outlet.

Item 25. The device of any one of items 1-9, 13-18, or 20-24 or the method of any one of items 10-24, wherein if the at least one fluid transport channel has varying cross-sectional area as a function of position along the channel length, at least one fluid transport channel tapers along its entire length.

Item 26. The device of any one of items 1-9, 13-18, or 20-25 or the method of any one of items 10-25, wherein if the at least one fluid transport channel has varying cross-sectional area as a function of position along the channel length, the channels taper along a portion of their length.

Item 27. The device of any one of items 1-9, 13-18, or 20-26 or the method of any one of items 10-26, wherein the voltage applied to the single molecules passing through each fluid transport channel is the same.

Item 28. The device of any one of items 1-9, 13-18, or 20-27 or the method of any one of items 10-27, wherein the voltage applied to the single molecule does not change during an experimental run.

Item 29. The device of any one of items 1-9, 13-18, or 20-28 or the method of any one of items 10-28, wherein one or more of the following variables are adjusted to optimize the duty cycle: applied voltage, applied current, applied fluid pressure, applied vacuum, applied centrifugal force.

Item 30. The device of any one of items 1-9, 13-18, or 20-29 or the method of any one of items 10-29, wherein the method employs a detection system that does not move during the detection step.

Item 31. The device of any one of items 1-9, 13-18, or 20-30 or the method of any one of items 10-30, wherein the method employs a detection system that does move during the detection step.

Item 32. The device of any one of items 1-9, 13-18, or 20-31 or the method of any one of items 10-31, wherein the single molecule flows through the detection region only one time.

Item 33. The device of any one of items 1-9, 13-18, or 20-32 or the method of any one of items 10-32, wherein the single molecule flows through the detection region multiple times.

Item 34. The device of any one of items 1-9, 13-18, or 20-33 or the method of any one of items 10-33, wherein the single molecule flows through the detection region multiple times due to oscillatory actuation.

Item 35. The device of any one of items 1-9, 13-18, or 20-34 or the method of any one of items 10-34, wherein oscillatory actuation is achieved by reversing polarity of the fluid transport channel.

Item 36. The device of any one of items 1-9, 13-18, or 20-35 or the method of any one of items 10-35, wherein the analyte is combined with a detection aide either before or after introduction into the device.

Item 37. The method of any one of items 10-36, further comprising dislodging a clog of the analyte or the single molecule in the fluid transport channel.

Item 38. The method of claim 37, wherein the clog is dislodged by applying an electrophoretic voltage to the fluidic transport channel temporarily or by illuminating the fluid transport channel with a high-intensity light source.

Item 39. The device of any one of items 1-9 or 13-15 or the method of any one of items 10-38, further comprising performing an assay, wherein the assay conditions are controlled via the adjustments to the duty cycle.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

What is claimed is:

1. A fluidic chip for detecting an analyte comprising:
a. at least one device comprising
i. a fluid inlet and
ii. at least one fluid transport channel that passes through a detection region, wherein (1) the at least one fluid transport channel has varying cross-sectional area as a function of position along the channel length and/or (2) the device comprises two or more fluid transport channels configured with two or more different cross-sectional areas; and
b. at least one fluid outlet,
wherein the device provides for transport of single molecules across the detection region at a desired duty cycle by (1) allowing for detection of an analyte at a different position along the channel length based on the desired cross-sectional area and/or (2) allowing for detection of an analyte in a specific channel configured with the desired cross-sectional area.

2. The fluidic chip of claim 1, wherein the duty cycle comprises a desired rate of detection.

3. The fluidic chip of claim 1, wherein the duty cycle comprises a desired spacing between molecules.

4. The fluidic chip of claim 1, wherein the cross-sectional area within at least one fluid transport channel varies as a function of position along the channel length.

5. The fluidic chip of claim 1, wherein the cross-sectional area within at least one fluidic transport channel is constant.

6. The fluidic chip of claim 1, wherein at least one fluid transport channel is a macrochannel, millichannel, microchannel or nanochannel.

7. A method of detecting an analyte using a fluidic chip comprising:
providing the fluidic chip of claim 1;
wherein (1) if the at least one fluid transport channel has varying cross-sectional area as a function of position along the channel length, detecting an analyte at a different position along the channel length based on the desired cross-sectional area in order to optimize duty cycle, and/or (2) if the device comprises two or more channels configured with two or more different cross-sectional areas, detecting an analyte in a specific channel configured with the desired cross-sectional area in order to optimize duty cycle.

8. A method of detecting an analyte using a fluidic chip comprising
a. providing a fluidic chip comprising (1) at least one device comprising a fluid inlet and at least one fluid transport channel that passes through a detection region and (2) a fluid outlet;
b. adjusting the rate of transport of single molecules through the fluid transport channel across at least the detection region by:
i. adjusting actuation energy applied to the analyte;
ii. electro-osmotic force tuning;
iii. tuning of ionic strength and/or mobility; or
iv. a combination of two or more chosen from (i), (ii) and
wherein adjusting the rate of transport of single molecules yields a desired duty cycle.

9. The method of claim 8, wherein the actuation energy is controlled by at least one of voltage, current, pneumatic pressure, vacuum pressure, flow focusing, and/or centrifugal force.

10. The method of claim 8, wherein transport of single molecules across the detection region is achieved by:
a. electrodes capable of applying a voltage or current difference integrated with the chip, optionally the electrodes being placed in contact with liquid in or on the chip;
b. pneumatic adapters to apply pressure or vacuum integrated with the chip, optionally the adapters being on fluid reservoirs of the chip;
c. rotation of the chip and optionally other system components to generate centrifugal forces; or
d. a combination of two or more chosen from (a)-(c).

11. The method of claim 8, wherein the adjustments to the duty cycle are based on pre-run factors.

12. The method of claim 8, wherein the adjustments to the duty cycle are based on closed loop feedback during an experimental run.

13. The method of claim 8, wherein the adjustments to the duty cycle are based on post-run analysis.

14. The method of claim 8, wherein the post-run analysis leads to culling a portion of the dataset.

15. The method of claim 12, wherein the experimental run is conducted under multiple conditions and post-run one or more of those conditions is selected based on optimization of duty cycle.

16. The method of claim 15, wherein conditions are selected based on optimization of duty cycle and those conditions are implemented using a different device.

17. The method of claim 8, wherein the adjustment to the duty cycle is based on
a. spike density above threshold;
b. time averaged signal;
c. likelihood of adjacent spikes confounding each other in a given run time;
d. ability to identify and cull individual confounding spikes from a dataset; or
e. a combination of two or more chosen from (a)-(d).

18. The method of claim 8, wherein if the at least one fluid transport channel has varying cross-sectional area as a function of position along the channel length, the cross-sectional area is smaller closer to the inlet and the cross-sectional area is larger closer to the outlet.

19. The method of claim 8, wherein if the at least one fluid transport channel has varying cross-sectional area as a function of position along the channel length, at least one fluid transport channel tapers along its entire length.

20. The method of claim 8, wherein if the at least one fluid transport channel has varying cross-sectional area as a function of position along the channel length, the channels taper along a portion of their length.

21. The method of claim 8, wherein one or more of the following variables are adjusted to optimize the duty cycle: applied voltage, applied current, applied fluid pressure, applied vacuum, applied centrifugal force.

22. The method of claim 8, wherein the single molecule flows through the detection region multiple times due to oscillatory actuation.

23. The method of claim 8, further comprising dislodging a clog of the analyte or the single molecule in the fluid transport channel.

24. The method of claim 23, wherein the clog is dislodged by applying an electrophoretic voltage to the fluidic transport channel temporarily or by illuminating the fluid transport channel with a high-intensity light source.

25. The method of claim 8, further comprising performing an assay on the analyte, wherein the assay conditions are controlled via the adjustments to the duty cycle.
